(19) United States
(12) Patent Application Publication
(10) Pub. No.: US 2008/0300571 A1
(43) Pub. Date: Dec. 4, 2008

(54) PROCESS AND DEVICE FOR SELECTIVELY TREATING INTERSTITIAL TISSUE

(76) Inventor: Patrick LePivert, Jupiter, FL (US)

Correspondence Address: MCHALE & SLAVIN, P.A., 2855 PGA BLVD, PALM BEACH GARDENS, FL, 33410, US

(21) Appl. No.: 12/129,138

(22) Filed: May 29, 2008

Related U.S. Application Data
(60) Provisional application No. 60/932,180, filed on May 30, 2007.

(51) Int. Cl. A61M 31/00; A61M 29/00

(52) U.S. Cl. 604/503; 604/506; 604/96.01

(57) ABSTRACT

A method and apparatus for direct interstitial treatment of tissue, while preventing backflow and decreasing drainage of a flowable composition from an injected area, by using a catheter and/or a needle with single or multiple inflatable member(s) that stretches, dilates and compresses target tissue and allows for an improved interstitial deposition, distribution and retention of the flowable composition, drug(s), agent(s), or particle(s), into a body organ, fluid, tissue, or tumor, thereby increasing procedural safety and efficacy of direct interstitial therapies.

### REFERENCE TO RELATED APPLICATIONS

This application claims benefit of the filing date of Provisional Application 60/932,180, filed on May 30, 2007, the contents of which are herein incorporated by reference.

### FIELD OF THE INVENTION

The present invention relates, in general, to fluid delivery devices and methods for delivering drugs to interstitial tissue structures within the body, more particularly, to new and useful devices and methods for delivering active agent(s) to selected sites of a tumor tissue, and most particularly to delivery of cytotoxic agents to a particularly problematic region within a tumor which has resisted more conventional therapy modalities.

### BACKGROUND OF THE INVENTION

The direct introduction of active therapeutic agent(s) or composition(s) such as a drug, compound, contrast agent, biologically active peptide, gene, gene vector, protein, or cells, into the tissues or cells of a patient in need thereof, can be of significant beneficial value. Tissue injection has long been a popular, relatively non-invasive means for the direct introduction of various medicaments and other fluids and is becoming more popular as a means for relatively non-invasive delivery of pharmaceutical preparations of cytotoxic drug(s), into solid tumor because it minimizes tissue trauma, increases local efficacy and decreases side effects and systemic toxicity. Direct injection, using needle, catheter, combined deposition system, and the like is also a practical delivery strategy for antiangiogenesis, tumor embolization, hemostasis, and direct cell kill.

Direct interstitial chemotherapy is slowly gaining ground among the medical community. A growing number of research papers and clinical reports published during the years 1990-2000 have shown improved efficacy and reduced toxicity in animal as well as in human tumors (E. P. Goldberg, 2001). However some studies showed an increased number of metastases with intra-hepatic chemotherapy versus i.v. treatment (Chang A. E. et Al. Ann Surg 1987)

New drug formulations allowing for time released or controlled release delivery of drug(s) to their target are now being made available to clinicians which extend the possibilities already available by the use of free drugs. Polymer gels used as drug carriers like 5-FU-polymer (Matrix Pharma MPI5003) or cisplatin-epinephrine gel (Matrix IntraDose) have been shown to be efficient against basal cell carcinoma—BCC—and hepatocellular carcinoma—HCC—(complete response and remission). Most importantly such intralesional injections demonstrated tumor concentration of drug being 10 to 100 times higher than for systemic chemotherapy, although the total intralesional dose was 10 to 32 times lower than for systemic chemotherapy (cisplatin-epinephrine gel for head and neck highly refractory solid tumors-Agerwall 1998). On certain far-advanced disease, i.e. painful bone or soft tissue metastases, a significant improvement in patient quality of life was observed. It has been recently suggested that neo-adjuvant direct—i.e. intratumor—chemotherapy could be a new treatment modality of great interest since systemic neo-adjuvant chemotherapy has already been proven very valuable on different types of solid tumors such as breast, lung, prostate, and colon cancers (see Goldberg). Direct injection of active agent(s) is also a very promising potential for controlling such aggressive lesions as brain tumors or such benign growth as prostate adenoma, a highly prevalent lesion in the US population.

Recent application of per-endoscopic direct intratumoral (IT) chemotherapy (Celikoglu, 1997) for endobronchial obstructive tumors has shown a relief of airway obstruction in a majority of patients without side-effects. The authors consider this treatment as a cost effective palliative modality. They have also combined IT chemotherapy with external radiation therapy with encouraging results on first hand non-operable endobronchial lesions.

Local electrochemotherapy has been proven more effective when the drug was administered interstitially rather than intravenously. Such active agents as saline solution, heating or cooling fluids, photosensitizer, radiosensitizer, radioisotopes, sclerosants (sclerosing agent), radioseeds, thermoseeds, glue, vaccine, gene, immunologic factors, hormones, particles, nano particles, combination and/or formulations can be injected.

The instantly disclosed method uses controlled tissue stretch and compression to direct the flow of directly injected compositions towards and/or away from selected target areas. In lieu of being injected locally, active agents may be injected systemically, i.e. intravenous or regionally, intra-arterially, or into other body cavities (like peritoneum, pleura, etc. . . . ), while the tumor is selectively compressed. Moreover, direct injection along with regional and/or systemic injection can be used when indicated.

The combination of elective pin-point stretch-compression of hypoxic areas of tumor, like that found in center of tumor, make the treatment potentially useful in combination with radiation therapy and/or initially radioresistant tumors. By stretching the hypoxic tumor cells outwardly the expanded member gets them mechanically stressed and more sensitive to radiation therapy. Moreover if the injected cytotoxic agent(s) is a radiosensitizer, the deleterious effect can be synergistic with radiotherapy. Precise guidance and placement of the stretching inflatable member(s) can be obtained with all known imaging technologies like US, CT, MRI or fluoroscopy. Imageable coating of the catheter shaft or inflatable member and/or filling of the latter with echogenic and/or radio opaque medium will help in detection and placement of the expandable delivery member.

The delivery strategy of administering cytotoxic drug(s) directly into a tumor is a major issue limiting its widespread use. For any therapeutic agent to be effective, it must accumulate in target cells in optimal concentrations for a required duration of time. In this regard sustained release or controlled release compositions (microsphere entrapment, matrix-based formulations, liposomes or polymer gels, etc. . . . ) seem more effective than free drug in that they allow prolonged intratumoral drug residence time. However these usual depot approaches deliver drug through the interstitial space by diffusion, which limits tissue penetration to a few millimeters. More recently, development of specific drug formulations, such as water miscible organic solvent vehicles for drugs, have provided for a fast and thorough saturation of the targeted tumor tissue, although such methods of treatment still require a homogeneous deposition of the fluid into the tumor.

The mixing of free drug and time-release formulation would provide an immediate beneficial effect as a result of the high drug concentration achieved on critical target components such as endothelial cells and tumor cells, leading to vascular thrombosis, tissue ischemia, and necrosis and durable effect of sustained lower concentrations of drug(s) on tumor cells leading to apoptosis and death.

Unfortunately, physicochemical and physiological barriers can lead to heterogeneous accumulation of various therapeutic molecules, particles, and/or cells in solid tumors. For instance it is well known that interstitial fluid pressure (“IFP”) is higher in most tumors than in normal tissue, related to tumor stiffness, and much lower at tumor margin (“TM”). Consequently fluid flow and transport of macromolecules by convection is poor in a tumor's center and wash out of agents is increased at the tumor margin, where pro-angiogenic activity is usually high. The wash out mechanism decreases the time of exposure of the TM to the active agent. TM is a known active and proliferating tumor zone. As a consequence one can expect a likely diminution of the agent's therapeutic efficacy. Moreover, the injection technique is very operator dependant in that it relies on perceived tumor margins and mass, subjective assessment of the number of sites of puncture to cover the entire visible mass, and subjective assessment of the fluid-dose fraction of active agent to inject at each site.

The operator, who usually relies on a commercial needle or catheter, must inject each site with a fraction of the calculated dose at a rate that is supposed to insure a “homogeneous” distribution/deposition of the agent(s) about the needle tip. The needle tip should be kept steady during injection to avoid puncturing or injecting unwanted structures. Most often, the needle is pointed and with a single orifice, which doesn't allow for a controlled distribution of agent, being a fluid or a particle within the interstitial medium of the target. The tumor vasculature should be spared, as well as the necrotic zones of the tumor. Therefore, agent distribution after direct injection within tumor is random and based on uncontrollable convective forces, on perceived tumor “capacitance” for fluid absorption, (subjective fill counter pressure, late visualization of fluid backflow that most often cannot be prevented). Similarly, complete dose administration isn't sure since backflow cannot be prevented, and is often quite difficult to assess, and intravascular administration isn't easy to detect. Moreover, multiplying the sites of injection increases chances of injury to risky tumoral structures, as well as operative time. Such conditions can lessen procedure tolerance by the patient with a risk of lesser patient compliance to repeated procedure.

The tissue delivery technique commonly used to homogeneously depose fluid or fluid-like agent(s) into an entire lesion has been to repeatedly insert multiple needle tips into the tissue to increase the diameter of induced necrosis/apoptosis. This approach, however, is both time-consuming and difficult to employ in the clinical setting particularly because multiple overlapping treatments must be performed in a contiguous fashion (in all 3D) to distribute agent to the entire lesion. Simultaneous use of multiple needles can reduce the duration of application but can be technically challenging in narrow passages or during endoscopic use. The development of multi needle injection devices with multiple arrays should enable the creation of larger foci of more homogeneous fluid distribution with a single penetrating site. Such features are dependant for their efficacy on the conditions of fluid transport existing in the target at the time of injection, and due to the extreme variability of these conditions, the distribution and deposition of active agent(s) at the right place cannot be predicted. Therefore, there is an unmet need for a predictable and uniform distribution of the active agent(s) over a selected tumor area.

Another issue with injecting fluid into tissue while preventing backflow with specific features, as described in U.S. Pat. No. 6,203,526 to McBeth, is that there is a need for a sufficient pressure to allow the flow of fluid out of the delivery openings of the instrument. Such pressure increases the risk of metastases by pushing migrating cells within the tumor vascular network, “mosaic vessels”, through which it is estimated that 1 million cells travel per day and per gram of tumor. The same issue arises with any delivery device that injects fluids or compositions into tumors.

Therefore there is also a need for a system that would allow for minimizing or preventing the unwanted migration of tumor cells off the bulk of tumor during the creation of elevated injection pressure.

In a disease such as cancer, failure to treat a small fraction of cells can result in tumor regrowth. Thus, it is crucial to know which tumor areas have been treated and which have not.

There are several steps which must be followed in order to insure a homogeneous exposure of targeted cells to therapeutic agent(s).

The first step is to use delivery systems that improve drug delivery and distribution to all regions of a tumor. It is recognized that solutions of free drug (ethanol, acetic acid, hypertonic saline etc. . . . ) as well as gel preparation of same drug (s), do not spread predictably and regularly through tissue (such as liver metastases and prostate cancer injected with ethanol or acetic acid). Single and multiple needle devices have been designed to overcome this obstacle (PROSTAJECT, etc. . . . ) that make use of pre-bent hollow needles. So far, they have met only limited success.

The second step is to make sure that the intended and whole dose of active agent has been delivered to the targeted site(s). Many techniques of injection, most of them calling for multiple passes of the needle for complete tumor coverage, have been used that meet with the observation of back flow of the injected fluid along the entry path of injection, either during or after injection, or along previous instruments/interventions paths, or at paths of low resistance or leaking structures like tumor vasculature. As a consequence, there is no assurance that the dosing is right and that the fluid has spread to the targeted regions and that exposure of surrounding healthy tissue to agent is minimized. For instance during trans-bronchial needle injection of lung cancers in end-stage patients with refractory disease there is a risk of spillage of the injectable agent into the surrounding airways and lung, the consequences of which depend mostly on the amount and toxicity of the agent being injected.

The third step is to avoid multiple needle sticks and to create a larger area of ablation at a single step than that which is usually observed with current needle injection techniques.

To circumvent these issues U.S. Pat. No. 6,203,526 B1 to McBeth shows a delivery device with a dam that expands radially and outwardly and prevents backflow of composition through the tissue track of the instrument for prevention of adverse effects of compositions on healthy tissue in the tracks of surgical devices. However there is no control over the distribution of the composition into the target, which is delivered into the central area, and no tissue distension-compression to minimize washout, minimize target volume and increase retention of composition.

U.S. Pat. No. 6,989,004 B2 to Hinchliffe shows radially deployable fluid members relative to an elongated axial penetrating member that allow for providing larger and more uniform needle treatment zone. However, after delivery of fluid-based drug(s) etc. . . . the transport of substances is determined by tumor characteristics that contribute to convection and diffusion, out of any instrumental control.

In US application publication # 20050273049 A1 to Krulevitch a drug delivery device using two concentric inflatable members is described. The delivery of fluid to a precise depth is affected by microprojections that create channels into target tissue. Such a device would increase the risk of bleeding and flushing of tumor cells resulting in migration and causing the tumor to possibly metastasize.

Generally speaking the use of single or multiple needle or microneedle injection systems are aggressive and risky for tumor structures drainage systems and since they build pressure during injection they increase the risk of spilling tumor cells through this fragile and leaking vascular network.

Also the use of balloon tipped catheters that are capable of delivering fluid to tumors under pressure are of three main configurations: porous wall of delivery balloon (no control over exact amount of composition delivered), concentric delivery sleeve or sweating balloon which allows for a precise dose delivery, or balloon-needle combination (which has the aggressiveness of the needle system).

Most devices are designed for intravascular and/or hollowed body tissue. U.S. Pat. No. 5,364,356 teaches a sleeve catheter that has a reconfigurable sleeve expanded by a balloon and that is taught as being usable for treating tumor tissue. However the '356 patent doesn't teach a device that would prevent backflow of fluid or how to configure a device for treating large tissue volume.

The use of anti-backflow systems alone during intratissue injection increases the risk of spilling migrating tumor cells and doesn't allow for directed flow of injected substances or for decreasing volume of tumor target to be injected.

Therefore there is a need for an interstitial injection device for a solid tumor treatment that would be able to inject non-aggressively and homogeneously a targeted area without increasing the risk of flushing tumor cells, and providing for control of backflow of injected composition through the entry track of the injector.

There is also a need for an interstitial injection device for a solid tumor treatment that would reduce the distance of the delivery device to the outer margin of tumor and that would have the potential to decrease the amount of active agent(s) necessary to successfully treat the targeted area.

Additionally, it is a point of major concern that tumor drainage through tumor and tumor margin vasculature be inhibited in the vicinity of the injection site(s) so that the drug(s), compositions, etc. . . . aren't immediately flushed out, and retention of drug(s) into site(s) of injection is prolonged.

It is therefore a distinct advantage of the instantly disclosed technology to compress the tumor stroma as well as its capillary bed, thereby decreasing the vascular drainage and wash out of the injected fluid(s)

Although the dosage of agent injected locally into tumor tissue, for instance during chemoablation, is usually much lower than that administered systemically for the same agent and indication, it may nevertheless be desirable that this amount be kept as low as possible to prevent any excess of composition to flow out of target and possibly harm healthy structures.

The presently disclosed method has the potential of keeping the dosing at a lower level than with previous devices by reducing the thickness of tissue to be treated. Moreover this method has the potential to make old and/or new, and or generic free drugs or compositions thereof very useful for the local, less expensive, treatment of cancer since the transport of drug(s) through a distended-compressed tumor, made denser, and momentarily deprived of vascular drainage in the vicinity of the delivery system is expected to be slower. With a precise placement of the compressing head(s) of the delivery system it is possible to deliver the cytotoxic agent(s) in the vicinity of the outer and peripheral margin(s) of tumor, which are well known for their ability to washout drugs and for their invasiveness.

It is also a major advantage that the compression-fluid delivery system of the invention has the potential for potentiating high concentrations of cytotoxic drug(s) like fluorouracil in the vicinity of the (inflatable member) balloon/tissue interface, resulting in interruption of protein synthesis and immediate kill of non-dividing tumor cells. It is well known that most epithelial malignancies have a majority of non-dividing cells.

It is also well known that spatial and temporal variations of tumor vasculature give rise to a shortage of blood supply with subsequent necrotic and hypoxic cells that exhibit a reduced sensitivity to ionizing radiation or to homogeneous spatial distribution of chemotherapeutic drug(s). The tumor stroma itself can be modified by neo-adjuvant radiation that may impair distribution of agent(s) into tumor.

It is therefore advisable that the operator makes injection preferably in metabolically active and proliferating (zone of tumor that may exhibit differences in temperature and conductivity, or other relevant parameters) parts of the tumor. It is also a major concern that the injection rate is preferably controllable and at adjustable pressure, and that the tumor vascular drainage be occluded during injection to avoid flushing tumor cells through the impaired tumor vasculature that could result in an increased risk of metastases occurrence (Chang et All, 1987).

In spite of the promise associated with most techniques of direct interstitial fluid-based therapies like chemoablation (indicated for such malignant tumors as lung, prostate, breast, head and neck, brain, and liver) and the potential clinical applications of these techniques, progress has been hampered by the lack of an effective means to achieve the overall objective of efficient and reliable therapeutic agent delivery using conventional techniques and devices. One of the most significant shortcomings of current systems is the inability to achieve reliable and consistent application from subject to subject. Significant sources of this variability are due to differences in the technique and skill level of the operator. Other sources of variability that are not addressed by current systems include differences in the physiologic characteristics between patients that can affect the application of the procedure.

Thus, even though it is desirable to maintain the dosage amount of agent injected locally into tumor tissue at a level which is much lower than that administered systemically for the same agent and indication, it is still desirable that this amount be kept as low as possible to prevent any excess of composition from flowing out of the targeted area and possibly harm healthy structures, create complications, or serve to prolong the procedure. The presently disclosed method has the potential of keeping the dosing at a lower level than possible with prior art devices by reducing the thickness/volume of tissue targeted for treatment.

Given that reliable and consistent application of clinical therapies of direct intratissular injection is highly desirable, the development of improved application systems is well warranted. Such development should include a means for minimizing operator-associated variability while providing a means to accommodate the differences in tumor and patient characteristics likely to be encountered during widespread clinical application of fluid-based interstitial therapies used as the sole treatment or in conjunction with loco-regional or systemic therapies.

### SUMMARY OF THE INVENTION

The present invention is directed towards instruments used to controllably and safely stretch, dilate, and compress tissue and inject drug(s), active substances or other compositions into a body organ or tissue or solid tumor at any location within the body and methods for their use. Fluid delivery devices and methods for delivering drugs to interstitial tissue structures within body are disclosed, along with new and useful devices and methods for delivering active agent(s) to selected sites of a tumor tissue. For example, the instantly disclosed method and device may be used for delivering cytotoxic drugs to a particular region within a tumor such as the tumor margin and/or within critical areas for growth and expansion, and/or to areas exhibiting resistance to conventional therapies.

The basis for the invention resides in the provision of a compression and fluid delivery system having at least an expandable member and a delivery lumen capable of delivering active agent(s), e.g. in the form of a cytotoxic agent(s) to a target tissue such as an endobronchial tumor, while minimizing the exposure of the healthy tissue, e.g. the bronchial tree, to the cytotoxic agent(s). Such methodology is capable of reducing the duration of an operation, reducing the number of applications, reducing the total dose administered, and increasing the safety and effectiveness of intratumoral fluid-based therapies.

In one embodiment, a method and device is taught wherein injection of a fluid composition is effected during balloon inflation. In a preferred, albeit non-limiting embodiment, tissue distension-compression can be executed first and injection can be performed during or after balloon deflation and decompression. Alternatively tissue injection can be executed first and followed by tissue compression/decompression. Thus, in its broadest sense, the invention contemplates contacting of the targeted tissue with the active agent at any point before, during or after the dilating, compression and stretching (distension/compression) step.

Furthermore, it is contemplated that these tissue compression and decompression cycles can be single or may be replicated in multiple cycles which are repeated during injection and executed by a manual or automatic device, in a manner similar to that used for angioplasty.

In a particularly preferred embodiment, the invention is directed towards treatment of a targeted tissue within an area of healthy tissue, and more particularly to an apparatus and method capable of delivering a variety of active agent(s) to a target tissue volume, such as a tumor located within a body organ or lumen or otherwise healthy tissue, with an enhanced and controlled spread and retention of the active agent(s) dosage.

The present invention is generally characterized in an apparatus with a body having inflatable and fluid delivery member(s) arranged to distend and compress tissue structure and to control the directional flow of a composition to a target. A minimally invasive interstitial compression injector system is provided that is capable of non-aggressively securing to a target and of exerting adjustable tissue distension and compression during the procedure, within an expandable member whose volume and shape is adapted to tumor characteristics and application.

In one embodiment, an expandable injection system is taught which comprises a syringe or pump system, inflatable member(s), needle or catheter and allows injecting interstitially, manually or automatically, precisely measured amounts of composition directly into a body tumor or tissue, while minimizing or preventing backflow of injected composition during the procedure and applying compression to tissue structure to minimize or prevent drainage of the composition.

A plurality of expandable instrument designs may be contemplated for creating advantageously shaped or diffused clouds, streams, (or jets) of medicament, composition, contrast agents or other fluid-based agents. Optional built-in sensing elements (sensors) allow for treatment monitoring and/or tissue characterization. The apparatus allows also for steady positioning-securing of a needle, catheter or the like during controlled tissue compression and allows for concurrent and separate use of other interstitial fluid injection therapies and/or interstitial mechanical or energy-based devices like thermal, sonic, light ablation, or electroporation, or to enable aspirating of fluid, tissue debris, etc.

The system allows also for controlled tumor distension and compression of the interstitial space and simultaneous use of therapies, external and/or systemic and/or loco-regional.

In most studies interstitial injection chemotherapy has been performed with needle, micro needle and/or catheter(s). The invention proposes to solve various issues associated with these instruments and methods. It is an object of the invention to provide a device and a method for treating target tissues, particularly lung tumors, by injecting a composition into the distended target tissue.

Accordingly, it is an objective of the instant invention to teach a device and a method for treating target tissues by injecting a composition while minimizing exposure of healthy tissue to the composition.

It is a further objective of the invention to provide an apparatus with a low profile design and smooth surface to enable penetration of tissue with minimal tissue trauma.

It is yet another objective of the invention to provide an apparatus with means for stabilization and steadiness before, during and after injection resulting in better injection procedure by the clinician, and minimization of the risk associated with unwanted displacement and injection of composition in unwanted tissue.

It is a still further objective of the invention to provide an apparatus with means to prevent or minimize backflow of composition along the tissue entry track of instrument or along previous pre-existing device tracks.

It is another object of the invention to provide an apparatus with means to prevent back flow of composition and to distend and compress one or more selected areas of target tissue so that the composition is more effectively delivered to areas identified as being critical targets for therapy.

It is another object of the invention to provide an apparatus with means to prevent back flow of composition and to directionally distend selected areas of target tissue so that lower amounts of the active composition can be more effectively delivered to compressed critical areas of the tissue.

It is yet another objective to provide an apparatus with means that directionally distend the target tissue and compress tissue structure so that the composition is more effectively retained within the target tissue.

Other objects and advantages of this invention will become apparent from the following description taken in conjunction with any accompanying drawings wherein are set forth, by way of illustration and example, certain embodiments of this invention. Any drawings contained herein constitute a part of this specification and include exemplary embodiments of the present invention and illustrate various objects and features thereof.

### BRIEF DESCRIPTION OF THE FIGURES

FIG. 1A. is a schematic representation of the catheter according to the invention with the inflatable members in their expanded position

FIG. 1B illustrates a catheter fully protruded from a guiding cannula, embedded into a target and with the inflatable members expanded.

FIG. 1C is a schematic representation of a catheter of the present invention having a central guide wire.

FIG. 2 is a cross sectional view of a catheter shaft above the proximal inflated member of FIG. 1

FIG. 3A-3L is a schematic representation of sample shapes of inflatable members usable for the present invention.

FIG. 4 is a schematic representation of an endoscopic catheter according to the invention positioned within a target with inflatable members expanded.

FIG. 5A is a schematic representation of a multifunctional single inflatable member according to the invention, embedded into a target and fully expanded.

FIG. 5B is a schematic representation of an example of a pattern of distribution of sensors on surface of an expanded inflatable member.

FIG. 6 is a schematic representation of the fast acting treatment of a target with two catheters simultaneously embedded.

FIG. 7A, FIG. 7B and FIG. 7C are cross sectional views of an asymmetrical inflatable distal member according to the invention embedded within an encapsulated tissue as it expands from a low profile to an inflated diameter.

FIG. 8 is a schematic representation of catheter according to the invention with an adjustable length of an inflatable distal member.

FIG. 9 is a schematic representation of a catheter according to the invention with an asymmetrical distal inflatable member expanded and acting in conjunction with an intra-arterial balloon catheter.

### DETAILED DESCRIPTION OF THE INVENTION

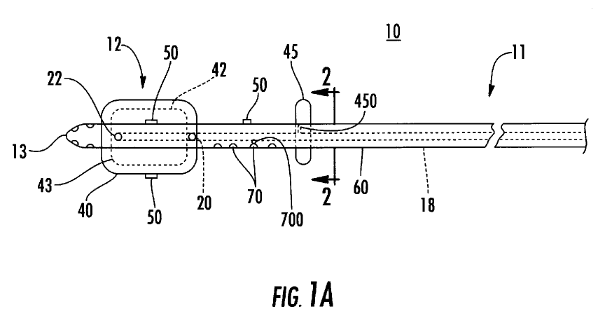

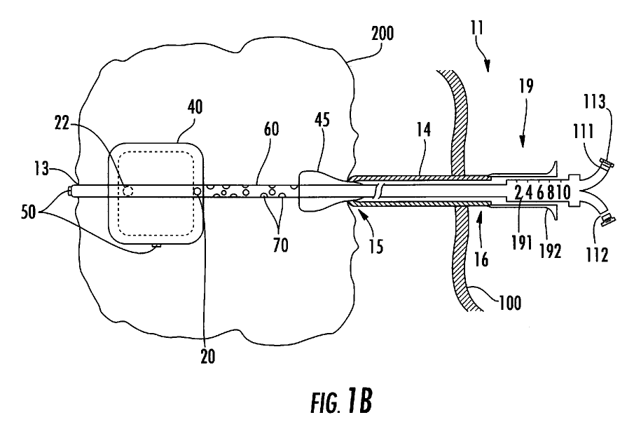

Methods, systems, and apparatus according to the present invention will rely on placement and use of one or more expandable element(s) positioned at or within a treatment region within the solid tissue of a patient. The treatment region may be located anywhere in the body where fluid injection may be beneficial. Most commonly, the treatment region will comprise a solid tumor within an organ of the body, such as the liver, kidney, lung, bowel, stomach, pancreas, breast, prostate, uterus, muscle, brain and the like. The volume to be distended-injected will depend on the size of the tumor. The treatment region may also be identified with sensors for sensing tissue parameters such as electrical impedance, temperature, pressure, and optical characteristics disposed at the distal end of the indwelling catheter. One or more sensor(s) may be used in any desirable combination and disposition over indwelling end 12 (FIG. 1) of catheter.

The present invention generally provides a fluid delivery system along with tissue compression and preferably a fluid delivery system for delivering cytotoxic drug(s) to interstitial space of solid tumor tissue. While the system can be used for a variety of purposes, the system is preferably used to treat soft tissue tumors.

With reference to FIG. 1A, the compression fluid delivery device includes a catheter member (10) with a proximal end (11) and a distal end (12) and an inner lumen (18) extending therethrough, and dilation members (40) (42) preferably disposed proximate to distal end (12) of the catheter (10). At least one dilation member (40) can be disposed proximate to the distal end 12 of the catheter 10. At least a single dilation member catheter must include means for occlusion of the instrument entry track into tissue. The catheter is comprised of a shaft 60 with an inner lumen assembly generally referred to as 18 comprising adjacent multiple lumens 181, 182, 183 and 184 as more clearly illustrated in FIG. 2.

The shaft 60 of the catheter member 10 and/or the dilation member inner and/or outer wall 40 are optionally equipped with sensing members 50 electrically coupled to a data logger (not represented) and a controller and monitoring system (not represented).

The catheter 10 has control means (inflation/deflation, injection) at the proximal end 11. The distal end 12 of the catheter 10 may be steerable by conventional means. Alternatively, the distal end may be of a flexibility, toughness, and bendability, different from that of shaft 60. The tip end 13 is preferably closed, blunt or tapered or round, but may be of any desirable shape, flexibility and openness like pointed, sharp, cutting, fully open, pre-bent, or bendable, and/or steerable. Such metal as stainless steel, or metallic alloy, or memory material like nitinol may be used for its structure. Any desirable combination of toughness, slidability, or flexibility and other desirable characteristics for the proximal end 11 and distal end 12 of catheter 10 is within the spirit of the invention.

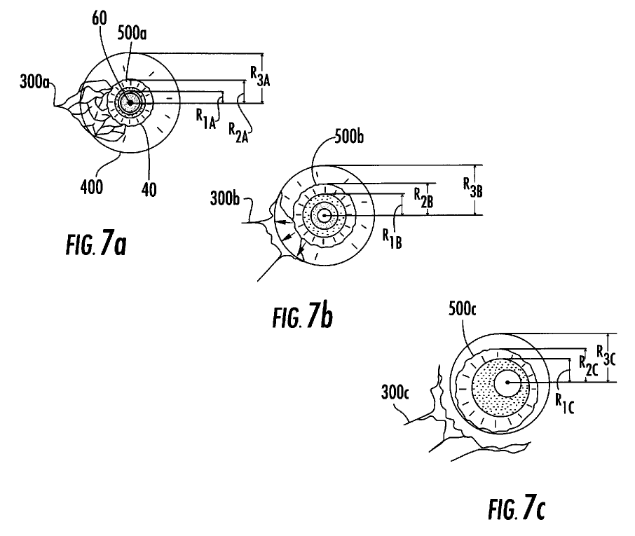

Proximal end 11 has an extension member 110 which comprises two separate tubing 111 and 112 of FIG. 4. Tubing 112 is coupled to inner lumen assembly 18 of catheter 10 and more particularly to inner lumen 181 that communicates with inner inflatable member 42 by opening port 20 located at distal inner end of inner inflatable member 42. Tubing 112 communicates also with second inflatable member 45, located proximal to inflatable member 42 along outer shaft 60 of catheter 10, by opening 450. Proximal end of tubing 112 of FIG. 4 is connected to an inflation/deflation system (not represented), such systems being well-known, that provides means to inflate and deflate at a controlled rate and pressure, the inner inflatable member 42.

Tubing 111 of proximal end 11 is coupled to inner lumen assembly 18 of catheter 10, and more particularly to inner lumen 182 that communicates through opening port 22 with interior 43 of outer inflatable member 40 concentrically disposed over inner inflatable member 42. Inner lumen 182 is also coupled with shaft openings 70 that are always located distal to inflatable member 45. Shaft openings may be distal and/or proximal to distal inflatable member 42 and/or delivery member 40. Tubing 111 is connected at its proximal side to a pressure fluid source and to a vacuum source through a manifold that allows composition delivery or aspiration into space 43 created between outer wall of inner inflatable member 42 and inner wall of outer inflatable member 40. For instance, a 4-way manifold (not detailed here) could allow for a multiplicity of operations such as aspirating fluid(s) and waste, injecting separately or in conjunction composition and/or other desirable agent(s) like contrast medium, hot or cold fluids.

Lumen 184 of shaft lumen 18 (FIG. 2) is optional and may be in fluid communication with a fluid pressure source and openings 70 when a separate fluid injection/aspiration through openings is desirable. Optional conduit 185 runs along shaft longitudinal axis within wall thickness of shaft 60 and may couple sensor(s) 50 with signal data logger(s) (not represented) through electrical wire(s), optical fiber, or any coupling cable for signal transmission known by those experts in the art.

Proximal end 11 of catheter 10 bears means 19 for adjusting depth of immersion of distal end of catheter 60 out of outer sheath 14 (FIG. 1 bis)

The inner lumen assembly 18 of shaft 60 of the catheter 10 comprises also a lumen 183 that opens into second inflatable member 45 which is located axially along shaft 60 and always proximal along shaft 60 in relation to the location of inflatable member 42. Inflatable member 45 expands radially and outwardly off longitudinal axis of shaft 60, having an expanded diameter at a specified pressure that can be lower, equal or higher than that of inflatable member 42. Lumen 183 runs along length of catheter 10 and couples inflatable member 45 and an inflation/injection system for member 45. Separate inflation/injection systems for inflatable member 45, for instance with lumen 183 and opening(s) may serve for delivery of a sealing composition, for instance cyanoacrylate, fibrin glue, PEG (polyethylene glucose) and/or other sealing substances known to those expert in the art, for permanently sealing the entry track during withdraw of the instrument from tissue.

The catheter and lumen are made of plastic, and or composite materials that are compatible with critical parameters such as pressure, profile, yield, and compatibility with composition, pushability, flexibility, kink and torque resistance, sterilization and the like. They should also be able to incorporate wires and microcomponents such as sensors or optical systems. A number of materials are available for the purposes that are usually made for balloon catheters applications and for thin wall miniature tubing. Illustrative, albeit non-limiting materials are PET, Nylon, PE (crosslinked and other polyolefin), Polyurethane, PVC, and composites such as braided polyimide tubing, can be found from companies such as HV Technologies, Inc (Trenton, Ga.) and Peebax (polyamide/polyurethane composite) balloon catheters at companies like Pan Medical Limited (UK). Other material like Teco, PTFE, PeeBax, Hytrel, Polyimide, and braided polyimide are readily available.

The inflatable members 40 or 45 of distal end of catheter 10 is preferably a balloon whose profile, material(s), compliance, diameter, shape, length and burst pressure are carefully selected to controllably dilate tissue to a selected diameter, optionally deliver composition within tissue and prevent backflow of composition during dilation. Balloon member(s) can be selected from a variety of available materials, shaped to conform to various tumor geometry and/or indications. Although a high-pressure, low compliance, thin-walled cylindrical balloon, symmetrically expanding outwardly about the longitudinal axis of the catheter distal end 12 (FIG. 1A) is a preferred design, any other design that would be deemed more suitable to a specific target is readily available from major catheter or balloon catheter companies.

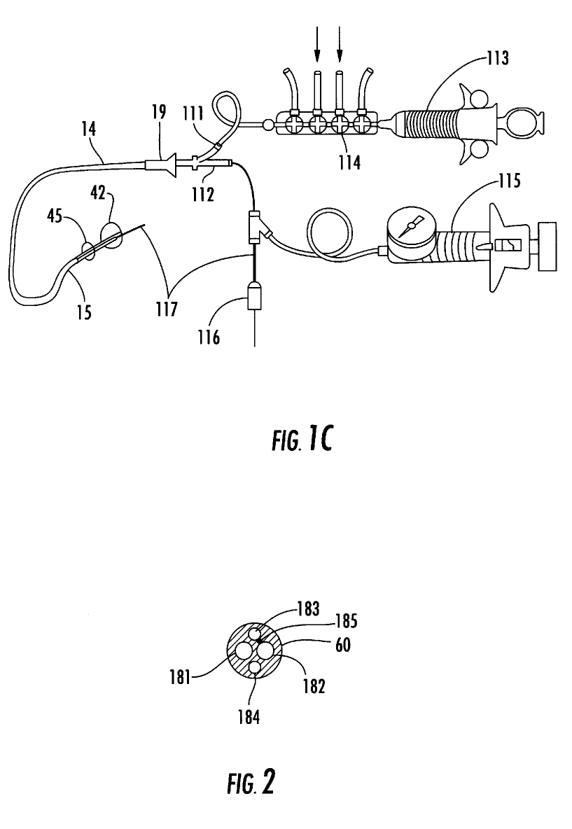

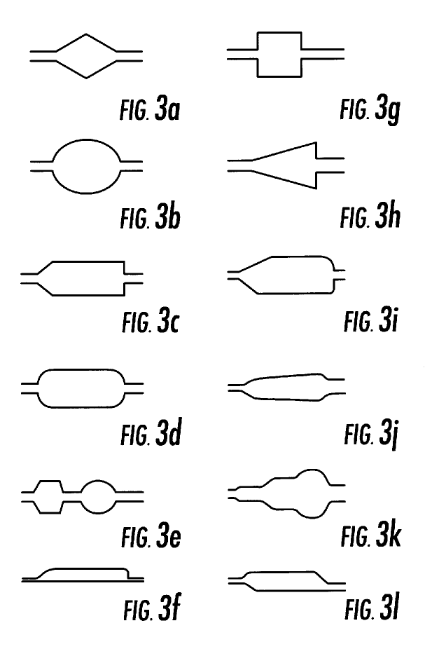

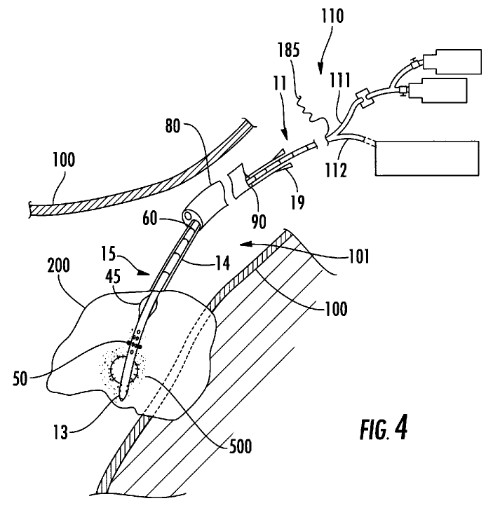

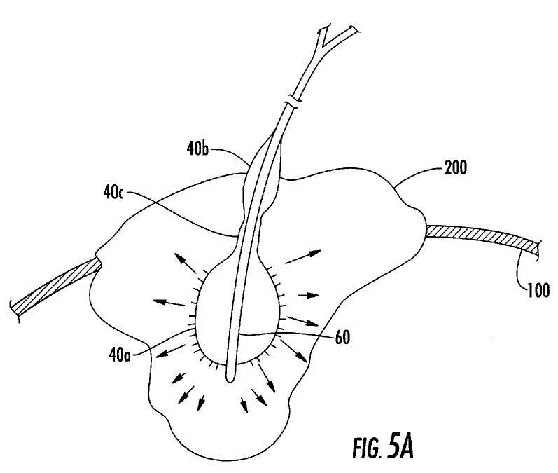

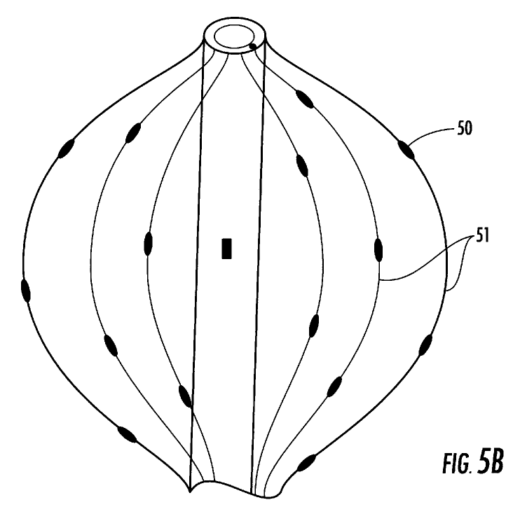

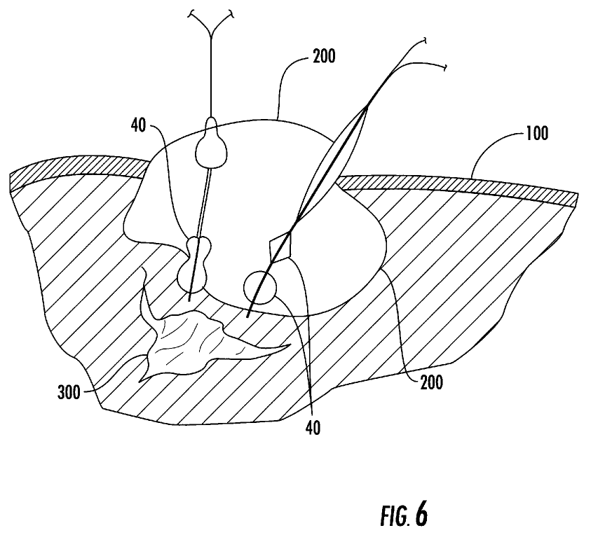

Balloon materials may be PET, Nylon, PE (crosslinked and other polyolefin), Polyurethane, PVC, and composite such as Peebax and the like. FIG. 3 shows balloon outer wall and tissue.

Balloon walls can be made of various shapes of balloon body, necks and cones, as exemplified, albeit not limited to the elements illustrated in FIGS. 3A-3L, that would fit most applications of controlled dilation of interstitial structures while being capable of delivering the composition at the interface between pressure elastomeric material, and such design may be selected preferably for the instrument entry track sealing member 45 which is represented in FIG. 1A.

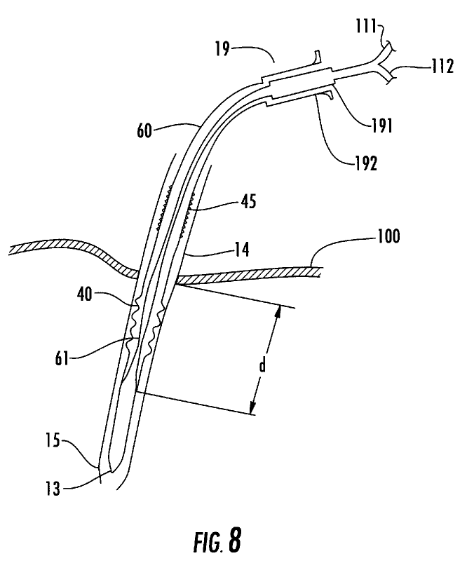

Although the preferred embodiment of (FIG. 1A) shows the dilation member 42 separate and distant from the instrument track entry occlusion member 45 both functions can be achieved by a single balloon having a specific shape such as a dog bone FIG. 5A. Such balloon may also have the capability of delivering the composition along a selected portion of their length and/or circumference through microholes, micropores and the like so that the delivery zones of the balloon do not compromise controlled tissue dilation and simultaneous occlusion of the entry track of the instrument. A dog bone balloon as represented in FIG. 5A delivers the composition through balloon 40 microporous surface of large diameter distal dilated section of the balloon 40a, while the impervious wall of the proximal section 40b of the balloon seals the entry track. Drug absorption and penetration into the tissue is controlled by the rate of fluid flow across the membrane and by the pressure at which the fluid is delivered. Fluid flow through tissue is controlled by the hole size and pattern. Alternatively the composition can be delivered through openings of shaft distal end 13 (FIG. 1A).

Referring again to FIG. 1A, the space between outer wall of inflatable member 42 and inner wall of outer member 40 is filled by composition and/or fluids to be injected into tissue through balloon permeable wall surface during, and/or at full inflation diameter of inflatable member 42. Such design of concentric balloons has the advantage over single balloon design to allow for the delivery of precise amount of composition volume. Following calculation of such amount, as described hereinafter the required effective dose is injected into space 43 through port 22 while balloon 42 is deflated. During inflation, and initiated by either a preset balloon 42 expanded diameter and/or pressure, the delivery of composition dose of space 43 will be effected to interstitial space of tissue through permeable wall of balloon 40.

The composition can also be delivered to tissue through openings 70 located along the circumference of shaft 60, and disposed distal and/or proximal to inflatable member 42 and/or distal to inflatable member 45.

Openings and/or microporous patterns can be of any desired design, symmetrical or asymmetrical about the longitudinal axis of catheter 10. The balloon can also be of any desirable shape and or symmetry related to longitudinal axis of catheter shaft 60. Openings can be of any desired size and either in fluid communication with fluid injection system of space 43 or connected to fluid pressure system of proximal end of catheter 11 through tubing 111 and through lumen 182 of shaft 60 or in fluid communication with a lumen 184 within shaft 60, connected at proximal end 11 of catheter with a fluid injection source such as a syringe.

The outer diameter of the shaft 60 could range in size, for example, from 0.3 mm for microprocedures, to 2.5 mm for endoscopic procedures, to 10 mm or more for open surgery procedure or direct procedures through natural openings or surgical cavities. The length of the shaft depending on the location of target tissue from the body surface could be in the range of, for example, 15 cm for easily percutaneously accessible tissue such as the prostate gland to 130 cm or up to 200 cm for endoscopic procedure to distal lesion of the arterial, aerial or digestive tract. In the illustrative embodiment, i.e. the apparatus for treating endobronchial tumor through a fiberscope, outside diameter of shaft 60 is 1.67 mm (0.066 inch), and outside diameter of sliding outer sheath 14 (FIG. 1B) is 2.7 mm (0.105 inch), maximal protrusion of distal end 12 out of outer sheath is 30 mm (5 mm to 70 mm), distal end diameter is 16 G (14 G to 21 G) and minimum channel size of endoscope must be 2.8 mm. Usable length of catheter, i.e. from distal end 15 to proximal end 16 of sheath 14, is 1700 mm. Proximal end 16 comprises a depth of penetration control means 19 that is made of a sliding stem 191 with a central lumen which has the same pattern of subdivision lumens of FIG. 2. Sliding stem 191 may be a thicker and rigid wall of catheter proximal part 11 whose advancement into outer rigid barrel 192 is controlled by mechanical pressure exerted by barrel 192 on stem of 191. Stem 191 bears marking that show depth of protrusion of distal end tip 13 of catheter 10 out of distal part 15 of outer sheath 14. Means for depth of insertion control are by no means limited to the system 19 and the advancement and penetration depth of a simple catheter without outer sheath could be controlled with a haemostatic valve located at the proximal entry of the biopsy (operative) channel 90 of an endoscope, provided that during immersion into target tissue the head of the endoscope be maintained steady.

There is a multiplicity of handles (Medi-Globe Corp., Wilson-Cook. Olympus., the list is not limitative) that can simultaneously luer-lock to the biopsy channel of the endoscope, secure the penetrating part 12 of the catheter 10 in retracted position within outer sheath 14 during sliding of apparatus into biopsy channel and towards target, secure outer sheath 14 when distal end 15 is contacting surface of tissue, secure advancement-positioning of dilation-injection tip 13 before inflation.

### Procedure:

For endoscopic uses it is important that the catheter be retracted into a protective and sliding outer sheath during guidance of instrument through biopsy channel of endoscope and towards tumor target. In the main embodiment of FIG. 1B the outer sheath is contacting surface of target tissue and maintained in this position by a securing means such as a haemostatic valve to the biopsy channel of the endoscope. Distal end 13 of catheter 10 has been set in right location at or near the tumor targeted margin and position has been assessed by imaging means and/or direct viewing. Inflatable members 42 and 45 are inflated at a selected rate and pressure determined (empirically) and based on patient pain, tumor characteristics and estimated compliance (among other variables) to variably or fully inflate balloons 42 and 45. Inflation of balloon 42 results in expansion of balloon 40 and balloon 45 is inflated at or near similar pressure. While the balloon 40 profile, i.e. diameter (inflation at specified pressure and shape) dilates selected parts of the tumor and compresses the tumor structure and vascular network, the diameter and shape of proximal balloon 45 seals entry track of apparatus by compressing the track walls. Given that track entry is usually not larger than outer diameter of apparatus, (balloon catheter has a low profile), and diameter of occlusion track balloon 45 is usually much smaller than that of the dilation member 40. However when a desired volume of inflation is to be distributed over a large tumor volume it may be advisable to use a larger diameter occlusion balloon or a multiplicity of smaller diameter balloons immersed simultaneously or sequentially into tumor (FIG. 6). It is understood by those experts in the art that any desirable combination of diameters, shape, and inflation pressure are possible depending on the operative conditions and desired effects. However since diffusion of composition through tissue depends much on differential pressure between balloon and hydrostatic/interstitial pressure of said tissue in contact with balloon walls and apparatus shaft, the balloon inflation pressure should be superior to the measured or estimated tissue pressure. Given that such pressure has been demonstrated as high as 100 mmHg (13 kPa, 1.9 psi) in tumor center, infusion pressure should generally be higher. Since elasticity of tumor tissue can vary considerably—from 0.9 bars to about 10 bars—the dilation pressure should be much higher particularly for hard, fibrous, or previously irradiated or otherwise treated tumor tissues. Therefore the pressure source should provide, and the apparatus shaft and expandable members should sustain from 1 to 15 bars of pressure, which means that burst pressure for the parts of the apparatus should be superior to the highest pressure required for effective functionality and safety of the apparatus. Pressures such as 1 to 20 bars could be used without departing from the spirit of the invention.

Given that injection may be given in certain circumstances at full inflation of expandable member 42-40 into space 43, injection pressure should be sufficient to overcome balloon/tissue interface pressure. Moreover expandable member 45 will be inflated at similar pressure to maintain seal of entry track during injection.

At full inflation and/or over threshold pressure for delivery of composition through micropores of balloon 40 the calculated dose injected into space 43 will be delivered to the interface balloon/tissue at a rate that is a function of the pressure and pore size and concentration. Simultaneously delivery of composition will be affected through openings 70 of shaft 60, according to the operator plan of treatment. It should be noted that dilation by inflatable member(s) entail, through circumferential and radial stress, a compression of surrounding tissue, which results in a zone of stress where capillaries are occluded, extra cellular matrix is denser, and entire surface of compressed tissue contacting balloon is exposed to composition. Such zone of target has the shape of the embedded inflated member. Another advantage of the invention is that the thickness of target tissue between inflated member and margin of tumor decreases, thereby providing potential for increased efficacy of composition that may reach tumor margin faster, at higher concentration, and with a prolonged residence time due to compression of drainage capillary network in the vicinity of inflated member. Provided that balloon and or shaft be imageable (echogenic coating, radiopaque coating or contrast agent into inflatable member 42 and/or mixed with composition, vibrating the system at the catheter's resonant frequency or any other suitable frequency), it is possible to image the shape and volume and immediate diffusion of apparatus and composition, with a potential for predicting the resulting tissue lesion shape and volume.

An additional advantage of apparatus according to the invention is that inflatable dilation member(s) may be positioned within tumor tissue region(s) that are known for their resistance to conventional therapies. For instance the center of a tumor is well known for being more radioresistant than more oxygenated peripheral regions of the same tumor. By precisely dilating such a zone of radioresistance and injecting radiosensitizer(s), such as fluorouracil (the list isn't limitative and well known by those expert in the art), the apparatus has the potential to treat successfully previous failure of radiotherapy.

An added advantage of the mechanical stress to tumor cells and stroma is that it entails a chain of events within stressed cells that lead to cell death through apoptosis. Since cytotoxic drugs act primarily on cell death through the triggering of apoptosis, the compression-chemotherapy method according to the invention has the potential for a synergistic powerful killing effect on cells at usual or lower than standard, i.e. minimal cytotoxic concentrations.

Another advantage of the apparatus for applying interstitial compression-chemotherapy to a tumor target is that the highest concentration of composition is delivered to compressed tissue. It's well known that selected drug(s) or composition(s) can entail immediate cell kill at higher concentration than those usually observed with systemic or even regional chemotherapy. Therefore the apparatus and method according to the invention has the potential to immediately relieve symptoms of obstructing or compressive tumors.

Now in the illustrative embodiment of FIG. 1B the distance between balloons is set to 5 mm, distal inflatable member 42-40 has a length of 7 mm and expands radially, outwardly and symmetrically about the longitudinal axis of shaft 60 up to a diameter of 10 mm. The shape is cylindrical. The length of proximal inflatable member 45 is 13 mm, shape is long spherical, and it expands radially outwardly and symmetrically about the longitudinal axis of shaft 60. It is understood that the length, shape, and/or distances separating balloons along the shaft 60 can be varied at will, provided that at least the entirety of distal inflatable member be immersed into target tissue (tumor, margin of tumor, and/or safety margin, and/or any desirable site of a target, either in plain or in naturally occurring or surgical/operative cavity of target), and that at least a length of proximal inflatable member 45 be engaged into entry track of apparatus (entry track being either a pre-existing track made by a diagnostic or operative instrument, the entry track of the apparatus into target, a naturally occurring lumen such as a vessel lumen, or may be an element of a guiding or positioning apparatus through which the apparatus of the invention passes to reach the target tissue.

The main embodiment of FIG. 1A shows a fixed distance—5 mm—separating inflatable members 42, 40 and 45. FIG. 1B shows that inflatable member 45 is fully immersed into target tissue so that after balloon 42 inflation and fluid/composition delivery through openings 70 and/or porous membrane 40, the entry track of the instrument is sealed to prevent backflow of composition through the track.

The composition amount to be injected should be calculated first and balloon dilation volume(s) should be added to determine safe procedural injection for compression-chemotherapy. As a rule, the dosing and inflation volume for a target tumor should be within a range of 20%-50% of calculated/estimated tumor target volume, but is not limited thereto. However, depending on particular operative conditions or requirements, the sum of injection+inflation volume may be varied, as well as the pressure at a specified inflation volume.

It is obvious that such composition volume delivery can be varied and augmented if the dilation sequence and the fluid delivery sequence aren't simultaneous, i.e. fluid delivery at full inflation, versus sequential delivery. That is, in the event when the target tissue is dilated for a selected period of time first, and after partial or full deflation of only the distal inflatable member 42 the composition delivery is performed through shaft openings 70.

Sensors located at various distances along the tip or shaft and monitoring systems are provided to measure significant tissue characterization and/or treatment parameters before, during and after injection (or Therapy). The parameters monitored may include the variation of bioelectrical impedance (Z) for estimating tissue hydration and conductivity (such as in breast cancer vs. breast tissue), where high conductivity reflects a highly metabolically active zone. Low conductivity reflecting necrotic or quiescent zones. Alternatively, elevation of impedance value may be used for monitoring ice ball (IB) growth (and size) through injection needle(s) during simultaneous use of cryosurgery. Additionally variations of bioelectrical impedance (Z) can be further monitored for assessment of injection effectiveness: Z (at variable frequency) which usually decreases during injection at under preoperative level and may be used for necrosis assessment; temperature of tissue for measuring zone of relative hypothermia (necrosis), characterization of hot or cold spot (thermal mapping) within tumor; interstitial pressure before, during or after inflation and/or injection; and white Light scattering (SpectraPath, Florida) for determination, characterization and location of cancer to healthy tissue margins during advancement of catheter or needle of the invention.

Another potential advantage of the method of “interstitial compression chemotherapy” permitted by the apparatus is that the amount of dose per injection per lesion—calculated on the volume of target tissue in the absence of interstitial inflatable member—would be reduced given that the volume of tissue to be treated is reduced by the inflatable member(s). In any case the injection of a reduced dose of composition at full inflation could be completed with another injection following full deflation of only the distal inflatable member 42 while the inflatable proximal member 45 is kept inflated.

It is contemplated that the sequences of inflation-injection-deflation-injection can be a single cycle or can be repeated as many time as needed in order to reach full saturation with composition and desired dilation of target. The rate of inflation, injection, deflation sequences and the time spent at a particular sequence of a cycle are adjustable and can be automated. FIG. 1C illustrates an over the wire catheter of the invention whose distal part 12 is protruding out of distal part 15 of a guiding sheath or cannula. Inflatable members are represented expanded. Flexible members of the apparatus can be replaced with a rigid metallic or plastic member so that catheter 10 and sheath 14 represented here become a needle-like inflatable instrument 10 and a rigid outer cannula or sheath or guide 14 (diameter 7F to 8F). Inflatable member 42 has an impervious wall and develops asymmetrically about the longitudinal axis of the catheter shaft to conform to tissue geometry. The composition dose delivery through openings 70 located between high compression noncompliant inflatable members 42 and 45 may occur in a procedure to treat a fibrous and hard lesion comprising various steps: step 1: guidance and insertion of guide wire into target using a pointed needle in case of a percutaneous procedure, direct or imaging assessment of placement of guide wire after needle removal; step 2: advancement of catheter 10 over the guide wire and pushing its distal end 13 towards tip end of guide wire. Assessment of positioning of inflatable members using marking and US or X-ray imaging of contrast material coating membrane of 42 and 45, or contrast agent injected into 42; step 3: knowing dose volume needed for target, initiating first treatment cycle, i.e. inflation of 42 and 45, injection of part or whole dose at full inflation of 42 and 45 through openings 70 and with the help of optional imaging (a mix of composition and contrast agent may be used to help in imaging composition diffusion); deflation of only 42 if added injection of composition is needed for total dosing, re-injection of the latter; a second or even a third treatment cycle may be performed whether based on tissue dilation and injection characteristics, patient's tolerance, and/or operator preferences.

Negative pressure is provided into space 43 and/or openings 70 through lumen 182 or optional lumen 184 if desirable. This allows for aspirating tissue fluid(s), or tissue structure components, for sampling or testing or pathology at any desirable moment during the procedure or for assessment of cell sensitivity at the end of the procedure. Aspiration allows also for relieving an excess of interstitial pressure occasionally resulting in a painful procedure for the patient or for increasing adherence of apparatus to tissue.

It is understood by those expert in the art that the catheter of FIG. 1B can also be used within the cannula 14. Cannula 14 is inserted through skin and pushed towards target either directly or guided by a central guide wire. Cannula 14 can penetrate target to a selected location and catheter is pushed through cannula towards targeted site.

FIG. 1C shows an embodiment of an over-the-wire design apparatus that comprises a central lumen within shaft lumen 18 of shaft 60 that allows for inserting a guide wire used to penetrate a hard lesion. Guide wire placement and location is detectable under ultrasound (US), CT, or fluoroscopic guidance. After placement at the desired location into tumor, the dilation-injection catheter is pushed over the wire down to its tip end.

Fluid injection source: FIG. 1C shows, as an example, a clear barrel and marking of a syringe to allow for precise dosage of composition during space 43 filling, and/or shaft openings 70 and pressure injection. Syringe 113 is luer-lock connected to a 4-way manifold 114, which allows for various fluid administrations.

### Mode of Injection: Manual or Automated:

The tip end 13 of the apparatus 10, is preferably closed, tapered, of variable length, and flexibility (or rigid). Alternatively the tip end may be absent and the distal end 13 becomes the location of the distal neck of the distal balloon 42 of the apparatus 10.

Alternatively the tip end 13 may be of any desirable flexibility, toughness, shape and even steerable if needed, designed with or without openings 70 for delivery of composition or aspiration of fluid(s).

Shaft 60 may be rigid, preferably of rigid plastic or a composite or metallic material such as stainless steel. The outer sheath or cannula or guiding tube may be of any desirable material, plastic or metallic, or composite. The shaft distal end 15 is preferably tapered and may be equipped with an inflatable member that could provide surface tissue compression or with a lumen built into the outer wall for the purpose of delivering a sealing agent(s) or any other desirable agent(s)

The outer balloon or sleeve or sheath structure 40, 45 can be made of any material having the desirable characteristics for the function of the invention. Besides the ultrathin walls (for minimal invasiveness and smaller profile), the porous or permeable walls, and the imageable coating previously described, other types of coating may be useful that would provide for a higher adherence of balloon surface to tissue when inflated, or provide increased resistance to abrasion. Such desired characteristics are well known from those who are expert in the art and aren't limitative, since almost any type of coating may be used provided that it is biocompatible with tissue and compatible with the structure's composition.

In the preferred embodiment of FIG. 1A and FIG. 1B, inflatable member 45 has two different shapes and volumes and is almost totally immersed into target and partially engaged into sheath 14 whose distal end 15 is in contact with the surface of tumor 200. Alternatively the inflatable member 45 may be oblong, cylindrical, with tapered ends or may have any other desirable shape and may be engaged totally or partially with the target and may also apply pressure and displacement on tissue structure.

In addition, inflatable member wall 45 can be set to deliver sealing agent(s) or a mixture of such at the end of the procedure to permanently occlude the entry track of the instrument. Among well known sealing substances are: cyanoacrylate glues, fibrin glues and the like.

In use, a target tissue must first be located and imaged for the purpose of assessing geometry and volume, relative location of risky or sensitive structures and estimating dose for direct injection. The manipulation of apparatus of the present invention to direct it to the target tissue may be effected by any suitable targeting and/or manipulation apparatus, method or procedure including instruments or elements separate from or integral with the present apparatus. The apparatus may be guided by means of any energy source, such as absorption, diffraction, scatter, or radiation, including any energy spectra (Infrared, X-ray, light), or CT, or MRI, or PET, or SPECT, or ultrasound. With reference to ultrasound guidance the apparatus may be made echogenic through an excitation device that oscillates the shaft and tip at selected frequencies. Such device is described in U.S. Pat. No. 5,967,991 assigned to EchoCath. As previously described the apparatus may be manipulated through any instrumentation approach modality like a cannula or trocar or guide wire or the like for instance for percutaneous approaches. Examples of targeting and/or manipulation devices, methods, or procedures that can be used alone or in combination with any tumor or anatomical structure of interest are further exemplified hereafter, and may include:

Endoscopic—e.g. for endoluminal and/or intramural, and/or extramural tumors or metastases of the bronchial tree or upper or lower digestive tract. An endobronchial approach with fiberscope instrumentation would allow for direct injection to endobronchial tumor or to reach extramural lesion or metastatic lymph node(s). Esophageal, rectal or colonic tumors can be reached with rigid or flexible endoscopes. Prostate tumor can be reached through urethroscopy.

Laparoscopic—e.g. for injection of liver tumors or of any abdominal organ like the pancreas etc.

Percutaneous for a multiplicity of target tumors like liver, pancreas, thyroid, lung, prostate, breast, spine, etc. . . . that can be reached using ultrasound guidance for needle positioning.

Open surgery: i.e. direct access to lesion through surgical open procedure where procedure can be applied in a tissue cavity left after surgery or made by the instrument of the invention.

For example, the fluid temperature that fills and inflate balloon 42 could be heated at sufficient positive temperature (40° to 60° C.) up to proteins and stroma main components denaturation. Such apparatus would have a return lumen in fluid communication with an additional port on shaft 60 open within balloon 42. A pump connected at catheter proximal end for instance to tubing 112 and to return tubing (not represented) from balloon 42 second opening port would circulate in closed loop the heated fluid, for instance a saline solution by a dedicated pump—not represented—Pump would include an electrical resistance and a heat exchanger or heater). Such thermo-distension of the tumor tissue would shape a cavity in the tissue by conformation of the heated stroma structure to the balloon shape resulting in a biological cavity. The delivery of composition into such cavity would have immediate effects and potentially could be a biological barrier to slow and delay diffusion of the active composition. A major advantage of the instantly disclosed system is that it provides for high local concentrations of drug(s), and longer residence time, which in turn may kill tumor cells that otherwise, would be (or could become) resistant to the drug(s). This feature paves the way for a revival of use of readily available free drug(s) with an enhanced local effectiveness and an absence of systemic effects. It is well known that a majority of epithelial cancer cells are at rest, therefore less susceptible to the action of cytotoxic composition(s) of free drug(s) which affect mostly rapidly dividing cells.

Alternatively, instead of releasing thermoseeds within a compressed tumor, balloon space 43 could be filled with those seeds. Balloon wall 40 would be impervious to seeds in this specific design and balloon interface would deliver controlled heat flow through a usually externally applied electromagnetic field. Many obvious advantages of this embodiment are: 1) that vascular flow occlusion will increase the thermal kill zone by reducing the cooling effect of drainage; 2) that tissue dilation heating can be made of any suitable size; and 3) that the apparatus is of very simple design without a need for any pump and fluid circulating system.

Alternatively, instead of being heated, close loop circulating fluid could be cooled, e.g. down to about −20° C. or lower, so that tissue surrounding the dilation balloon would at least reach hypothermic temperatures at distance from the interface tissue/balloon. In addition to entail microvasculature contraction hypothermia would stress tumor cells towards the apoptotic sequence and would act in synergy with cytotoxic drug(s) to enhance cell killing.

Balloon implantation: alternatively a low profile balloon catheter of the invention can be left in the tumor tissue or target for various purposes: follow up and sampling, imaging, continuous delivery of composition or as an adjuvant to conventional therapies, like external beam radiation, or locoregional or systemic chemotherapy. In this indication, the balloon would act as an adjuvant to better expose compressed tumor structures and cells to the energy source and/or agent(s). The apparatus of the invention could be temporarily left “in situ” with its inflation and injection ports 113 and 114 of tubing 111 and 112 (FIG. 1B) being either on patient's skin or out of a natural body tract, or lumen, or surgical wound. The apparatus of the invention could also be permanently left in situ and connected to small, portable subcutaneous implanted reservoir, pump and the likes.

The apparatus of the invention can be combined with any minimally invasive ablation techniques (HIFU, MW, Laser light, RF, cryosurgery, brachytherapy and the likes), or locoregional therapies (embolization, chemoembolization and the likes), or systemic therapies (chemotherapies)

The apparatus of the invention can also bear an energy source (HIFU, MW, Laser light, RF, cryosurgery, brachytherapy and the likes) within balloon(s) 42 and/or on its shaft 60.

Fluids and composition: it's impossible to cite all the agent(s), drug(s) composition(s), cell(s), tissue component(s), particles, nanoparticles, etc. that could be used with the apparatus of the invention. It will be obvious to those experts in the art that, for the purpose of tissue kill, compositions that act directly on cells and/or indirectly on tissue structure and more particularly on their vascular bed would be best suited since they would complete and reinforce the mechanical action of the compressive member(s). For example, agents such as Gelfoam, glue(s), absolute alcohol, epinephrine, cytotoxic drug(s) among others have vascular effects. Also (the list isn't limitative) drug, compounds, contrast agents, biologically active peptides, genes, gene vectors, proteins, or cells for therapy, into the tissues or cells of a patient can have significant therapeutic value. Thermoseeds, radioactive seeds, chemosensitizers, radiosensitizers (the list is not limitative) and the like can be used for combined procedures with the apparatus of the invention. Combinations and mixtures of the above mentioned agents or type of agents may be used without departing from the spirit of the invention. Likewise, the agents may be in the form of matrix-based, encapsulated or carrier based agents.

Now if the goal is to regenerate and or re-vascularize a tissue, the compressive stress strength and the duration of compression, as well as the microinstrument profile should be adjusted to allow for creating a microtrauma acceptable for revascularization through microchannels within ischemic tissue or tissue regeneration following cell and/or cell growth factors and agent(s) deposition.

### Monitoring Treatment and Procedure:

FIG. 1A. illustrates the location of sensor 50, located on the shaft 60 or on balloon 42 or 40 or 45 surfaces or interior. Such sensor would detect for instance changes in temperature, pressure, electrical impedance or light scattering of either the tissue in contact or the apparatus part to which they are attached. For example it is known that necrotic region(s) of a tumor has impedance that is different from that of vivid growing zones. By electrically mapping such region(s) it's possible to position the tip end 13 of shaft 60 at distance from necrotic region so that the inflated distal balloon would stretch primarily the necrotic region. As another example light scattering of cancerous cells is known to be different from that of normal cells so that it's possible to detect interfaces of tumor and normal tissue with a good precision. A tiny fiber optic cable built into the catheter and connected to a dedicated analyzer (Spectra-Path, Florida) would allow for positioning tip end 13 of catheter 10 at a critical region of a primary or recurrent tumor, i.e. the margin of tumor to healthy tissue that should be treated as a safety margin. Such built-in capability of optical characterization would allow for an added assessment of tip end location into target in addition to US imaging, or direct viewing.

If deemed necessary a multitude of sensors could be used in various arrays and patterns about the shaft and/or the balloon(s).

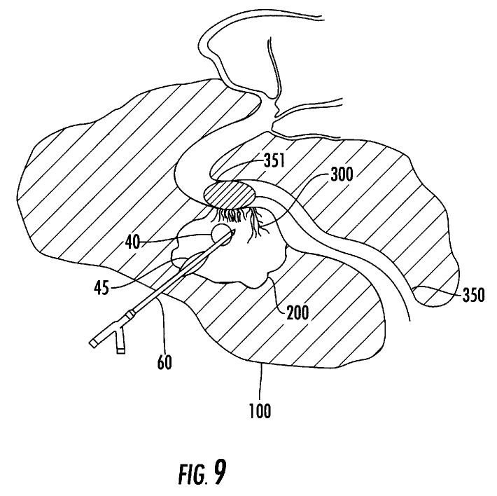

FIG. 7. illustrates the stretch and compression of an asymmetrically growing balloon 40 (radius R1a) about catheter shaft 60 (radius R1a) that results in a outwardly directional marginalization of tissue surrounding inflatable member 40. FIG. 7a shows a deflated balloon and catheter is located within an encapsulated tissue or tumor 400 (such as prostate). Tumor tissue is developing in a well vascularized peripheral area 300. Radial distance of center of cylindrical shaft 60 to tissue stretched and compressed by shaft is R2a, and radial distance of center of 60 to capsula is R3a. In FIG. 7b one sees that upon inflation of balloon 40 asymmetrically and preferentially towards 300b (arrows) increases radii R1b and R2 preferentially and more largely in direction of the vascular network 300b that is displaced and occluded. Now in FIG. 7c at full inflation tissue deformation is maximal towards 300c and distance between outer wall of balloon 40 and capsula 400 is minimal with almost only compressed tissue interposed. This region of large tumor/balloon wall interface is where the composition is to be delivered, which the apparatus of the invention allows.

FIG. 3A-3L. illustrates examples of various balloon shapes available for any interstitial application. Combinations of two or more of these basic shapes provide a way to design any desirable shape for symmetrical or asymmetrical, center or off center, expansion of balloon(s).

FIG. 4. illustrates a catheter of the invention with shaft 60 inserted within an outer sheath 14, and outer sheath 14 being inserted into the biopsy channel 90 of an endoscope (fiberscope). Distal part 15 of outer sheath 14 is positioned in contact with surface of tumor target 200, which is protruding off the wall 100 of an organ (bronchus, or esophagus for example) into the lumen of the organ 101. Tumor is partially eroding organ wall and invading outwardly through healthy tissue. Catheter distal end 12 is represented fully protruding out of sheath 14 and embedded into tumor target with tip end 13 located at distal margin of tumor. Drawing shows the estimated diffusion cloud of composition 500 about catheter distal part 12 from delivery through porous balloon 40, and shaft openings 70 distal to balloon 40 and to balloon 45. Inflated balloon 45 prevents composition from flowing back into entry track of apparatus. A means for providing a controlled depth of immersion of distal end 12 into tumor is represented with means 19 at proximal end of sheath 14. Barrel 192 can optionally be secured to biopsy port 90 of endoscope 80. Depth of immersion is assessed by distance markings of the catheter 60 and catheter position is secured by means 191 of catheter 10 that slide into barrel 192.

During guidance through endoscope, catheter 10 is maintained in retracted position into sheath 14 so that its tip end 13 doesn't damage the inner wall of biopsy channel. After completion of procedure and withdrawal of catheter distal end 12 from tumor, catheter distal end 12 is retracted into lumen of sheath 14. Sheath 14 is withdrawn from endoscope biopsy channel.

Depending on its design, sheath 14 can be reusable or disposable. Reusable outer sheath is preferably made of metallic spiral.

It must be noted that location of balloon 45 instead of being engaged partially or on its full length into entry track of instrument, may be located at surface point of entry track so that its inflation will occlude said entry track. An advantage of such location of the inflatable member 45 is to allow for an increased compressive stress of tissue interposed between inflated surface balloon 45 and inflated deep balloon 40. Such compression exerted between balloons could potentially speedup the treatment duration by superficializing deep tumor tissue. In such case balloon 45 would advantageously be cylindrical or with a large surface contact diameter with tumor.

FIG. 5 illustrates the simplest design of the invention where only a single expandable member 40 of fixed dimensions is used, where balloon shape is dog bone like with a distal diameter 40a larger than the proximal diameter 40b, with a distal shape more cylindrical and a proximal shape more oblong and a central narrow diameter segment 40c whose walls are impervious (continuous line). Balloon wall 40 is a porous dashed line—and allows composition delivery (arrows) over the large diameter part. Balloon 40 thereby is a stretch/compression device, and delivery device for the distal region of tumor tissue and an occlusion device for preventing backflow through the entry track. Any other shape that fulfill the single balloon device functions of tissue stretch compression, composition delivery, and track entry occlusion would be suitable for use. A needle like multifunction sensor is represented embedded into the tumor for monitoring tissue treatment (pressure, temperature, impedance, optical scatter). Sensors can also be mounted at surface of balloon(s) contacting tissue and can be lined along splines 51 with various configurations, such as a staggered configuration in FIG. 5B. Electrical components, wires that connect splines and sensors, or electrically conducive coating of outer balloon(s) and/or shaft surface, can be used as electrodes of an electroporation system for electrochemotherapy, or for delivery of RF energy. Composite material for balloon(s) and shaft may be preferably used for such multifunction apparatus.

FIG. 6 illustrates the simultaneous use of two catheters of the invention to expedite treatment of a large and easily accessible tumor, where more than two inflatable members may be used to insure a larger region of dilation compression while allowing precise placement of larger inflatable members 40 at target margin 200 of the distal end 13 of embedded catheters. Inflation of members 40 will compress vascular network and stroma 300.

FIG. 8 illustrates an embodiment of the interstitial compression delivery apparatus of the invention having an adjustable length of compressive expandable distal member 40. The central stem 61 that bears the lumen for inflation 181 and injection 182 can adjust its advancement into outer shaft 60 through the means 19. Means 19 allows for adjusting advancement and securing of plunger like part 191 into barrel like part 192 so that when 191 is fully pushed into barrel 192 balloon walls 40 are fully extended. A catheter is represented in retracted position into a cannula 14 that perforated tumor and allowed full immersion of distal part 15 into tumor tissue. After withdraw of cannula 14 catheter tip end 13 is positioned into tumor.

FIG. 9 illustrates the application of an embodiment for the apparatus according to the invention to a brain tumor. Needle like probe shaft 60 is introduced through the skull and the brain 100 to the deep seated lesion 200 that is adjacent to a large vessel 350 in which a balloon catheter 351 has been advanced to prevent drainage of active agents injected directly into tumor from escaping lesion, a well known procedure for those who are experts in the art. Now, a compression delivery device of the invention would have a similar effect on the vascular drainage 300 without the inconvenience and risks of intra-arterial selective or supra-selective catheterism. Moreover, the apparatus of the invention can be used in conjunction with any vascular catheterism device that intends to mitigate, or prevent drainage of a composition out of a tumor, and/or that is used for flowing a composition selectively into a tumor or a tumor bearing organ, and/or with any blood borne therapy that is used for selective trapping into tumor tissue, and/or with any therapy that is used for tumor targeted therapy.

All patents and publications mentioned in this specification are indicative of the levels of those skilled in the art to which the invention pertains. All patents and publications are herein incorporated by reference to the same extent as if each individual publication was specifically and individually indicated to be incorporated by reference.

It is to be understood that while a certain form of the invention is illustrated, it is not to be limited to the specific form or arrangement herein described and shown. It will be apparent to those skilled in the art that various changes may be made without departing from the scope of the invention and the invention is not to be considered limited to what is shown and described in the specification and any drawings/figures included herein.

One skilled in the art will readily appreciate that the present invention is well adapted to carry out the objectives and obtain the ends and advantages mentioned, as well as those inherent therein. The embodiments, methods, procedures and techniques described herein are presently representative of the preferred embodiments, are intended to be exemplary and are not intended as limitations on the scope. Changes therein and other uses will occur to those skilled in the art which are encompassed within the spirit of the invention and are defined by the scope of the appended claims. Although the invention has been described in connection with specific preferred embodiments, it should be understood that the invention as claimed should not be unduly limited to such specific embodiments. Indeed, various modifications of the described modes for carrying out the invention which are obvious to those skilled in the art are intended to be within the scope of the following claims.

What is claimed is:

1. A process for treating an interstitial tissue structure with an active agent comprising:
providing at least one catheter or needle having a proximal end and a distal end, a lumen assembly extending longitudinally from said proximal to said distal end, and at least one expandable dilation member distally located and adapted to be positioned at or within a treatment region within the solid tissue of a patient;
positioning said catheter distal end within or proximate to said treatment region;
expanding said at least one expandable dilation member, whereby interstitial tissue proximate said treatment region is dilated, compressed and stretched; and
contacting said active agent with interstitial tissue in said treatment region before, during or after expanding of said interstitial tissue;
whereby an enhanced and controlled spread and retention of the active agent is effectuated.

2. The process of claim 2 wherein said step of expanding simultaneously prevents backflow of said active agent.

3. The process of claim 1 further including providing a source of interstitial energy to said treatment region selected from the group consisting of thermal, sonic, light ablation, electroporation and combinations thereof.

4. The process of claim 1 wherein said step of contacting is selected from the group consisting of local injection, regional or systemic injection, insertion within body cavities and combinations thereof.

5. The process of claim 1 wherein said active agent is free or bound to a carrier to enable a controlled release, and is selected from the group consisting of saline solution, heating or cooling fluids, a photosensitizer, a radiosensitizer, a radioisotope, a sclerosing agent, radioseeds, thermoseeds, glue, a vaccine, a gene, an immunologic factor, a hormone, a cytotoxic agent, and combinations thereof.

6. The process of claim 1 wherein said interstitial tissue structure is a benign or malignant tumor, or a dysfunctional zone of an organ or target tissue.

7. The process of claim 6 wherein said interstitial tissue structure is a malignant tumor.

8. The process of claim 7 wherein said active agent includes at least one cytotoxic agent.

9. The process of claim 8 wherein said step of contacting said at least one cytotoxic agent with said dilated, compressed and stretched interstitial tissue includes delivering said cytotoxic agent to a particular region within said tumor including the tumor's margin, within critical areas for growth and expansion, and to areas exhibiting resistance to conventional therapies.

10. The process of claim 1 including a step of identifying said treatment region by using sensors for sensing tissue parameters selected from the group consisting of electrical impedance, temperature, pressure, optical characteristics disposed at the distal end of the catheter and combinations thereof.

11. The process of claim 1 wherein said steps of expanding and contacting are replicated in multiple cycles which are executed by a manual or automatic device.

12. The process of claim 6 wherein said steps of expanding and contacting are carried out while essentially preventing flushing of tumor cells and preventing backflow of said active agent.

13. An expandable injection system useful in the treatment of an interstitial tissue structure with at least one active agent comprising, in combination, a syringe or pump system, at least one inflatable member, and a needle or catheter constructed and arranged for interstitially contacting, manually or automatically, precisely measured amounts of said at least one active agent directly into a body tumor or tissue positioned at or within a treatment region within the solid tissue of a patient, while minimizing or preventing backflow of said active agent, and applying compression to said tissue structure to minimize or prevent drainage of said active agent.

14. The expandable injection system of claim 12 wherein said system further includes means for contacting said interstitial tissue structure with a source of interstitial energy to said treatment region selected from the group consisting of thermal, sonic, light ablation, electroporation and combinations thereof.

15. The expandable injection system of claim 14 wherein said active agent is selected from the group consisting of saline solution, heating or cooling fluids, a photosensitizer, a radiosensitizer, a radioisotope, a sclerosing agent, radioseeds, thermoseeds, glue, a vaccine, a gene, an immunologic factor, a hormone, a cytotoxic agent, and combinations thereof.

16. The expandable injection system of claim 14 wherein said inflatable member is at least one expandable dilation member, constructed and arranged to dilate, compress and stretch said interstitial tissue proximate said treatment region whereby an enhanced and controlled spread and retention of the active agent is effectuated.

17. The expandable injection system of claim 16 wherein said expandable dilation member is a unitary body which dilates, compresses and stretches said interstitial tissue proximate said treatment region while simultaneously preventing backflow of said active agent.

18. The expandable injection system of claim 16 wherein said expandable dilation member comprises a first occlusion balloon surrounding a second occlusion balloon, said occlusion balloons arranged concentrically and defining a dosage region for retaining said active agent therebetween, wherein expansion of said second occlusion balloon expands said first occlusion balloon causing it to dilate, compress and stretch said interstitial tissue proximate said treatment region, and wherein active agent inserted within said dosage region is pressurized and forced into said dilated, compressed and stretched interstitial tissue.

19. The expandable injection system of claim 18 wherein said first occlusion balloon is porous and said at least one active agent is released from the material by elution from pores.

20. The expandable injection system of claim 13 wherein said catheter has a proximal and a distal end, a central portion including a first longitudinally extending lumen assembly having at least one opening on its proximal end adapted for transport of said at least one active agent to said distal end thereof, and further including means for expansion of said at least one expandable dilation member.

21. The expandable injection system of claim 20 wherein said lumen assembly has a second longitudinally extending opening capable of communicating with said at least one expandable dilation member.

22. The expandable injection system of claim 13 further including sensors for sensing tissue parameters selected from the group consisting of electrical impedance, temperature, pressure, optical characteristics disposed at the distal end of the catheter and combinations thereof.

23. The expandable injection system of claim 13 wherein said means for interstitially contacting precisely measured amounts of said at least one active agent directly into said body tumor or tissue are adapted to replicate said contacting in multiple cycles.

24. The process for treating an interstitial tissue structure with an active agent in accordance with claim 1, further including a step of collapsing said expandable dilation member.

25. The process for treating an interstitial tissue structure with an active agent in accordance with claim 1, further including a step of retrieving and/or repositioning said catheter or needle.
